(19) United States
(12) Patent Application Publication
(10) Pub. No.: US 2013/0053730 A1
(43) Pub. Date: Feb. 28, 2013

(54) Miniature Sensor Tip for Medical Devices and Method of Forming the Same

(71) Applicants: Rama Krishna Kotlanka, Singapore (SG); Vaidyanathan Kripesh, Singapore (SG); Daquan Yu, Singapore (SG); Kok Lim Chan, Singapore (SG); Soo Yeng Benjamin Chua, Singapore (SG); Pavel Neuzil, Singapore (SG)

(72) Inventors: Rama Krishna Kotlanka, Singapore (SG); Vaidyanathan Kripesh, Singapore (SG); Daquan Yu, Singapore (SG); Kok Lim Chan, Singapore (SG); Soo Yeng Benjamin Chua, Singapore (SG); Pavel Neuzil, Singapore (SG)

(73) Assignees: National University of Singapore, Singapore (SG); Agency for Science, Technology and Research, Singapore (SG)

(21) Appl. No.: 13/504,455

(22) PCT Filed: Aug. 27, 2010
(86) PCT No.: PCT/SG10/00317

§ 371 Date: Nov. 9, 2012

(30) Foreign Application Priority Data
Oct. 30, 2009 (SG) 200907245-5

(51) Int. Cl. A61M 25/09

(52) U.S. Cl. 600/585

(57) ABSTRACT

According to embodiments of the present invention, a micro-sensory tip for use in blood vessels is provided. The micro-sensory tip includes: a force transmission element; at least three force detecting sensors coupled to the force transmission element, each of the at least three force detecting sensors responsive to force applied on the force transmission element, wherein each of the at least three force detecting sensors produces an output representing at least one force component of a three-dimensional Cartesian co-ordinate system, of the force experienced by the force transmission element, such that the outputs of the at least three force detecting sensors can cover the space of the three-dimensional Cartesian co-ordinate system; and an active element arrangement coupled to the at least three force detecting sensors, the active element arrangement configured to process the output from the at least three force detecting sensors.

### CROSS-REFERENCE TO RELATED APPLICATIONS

This application claims the benefit of priority of Singapore application No. 200907245-5, filed 30 Oct. 2009, the content of it being hereby incorporated by reference in its entirety for all purposes.

### TECHNICAL FIELD

Various embodiments relate to a micro-sensory tip for medical devices and a method of forming the micro-sensory tip.

### BACKGROUND

The ability to successfully treat a vascular lesion via endovascular methods (wires, catheters and angioplasty balloons) is dependent on the ability to pass a guidewire across the lesion (usually a stenosis or occlusion), generally known as the wire traversal test.

For successful passage of a guidewire through the narrowing vascular vessels, conventionally, a surgeon or an operator navigates the guidewire based on his or her own skill and haptic feel. Conventional techniques or systems do not provide force feed back and a number of such procedures are abandoned as the operator is afraid of exerting excessive force and piercing through the vascular vessels.

Blockage of the vessel lumen in the range from 50% to 100% makes passage of the guidewire a challenging affair. Up to 20% of endovascular procedures are abandoned due to failed wire traversal. In addition, a higher number of similar procedures are prolonged due to repeated attempts to manipulate the guidewire across the lesions.

Multiple and prolonged attempts at guidewire passage may lead to several undesirable side-effects, such as (i) increased exposure to radiation dosage due to prolonged fluoroscopy time, (ii) increased amounts of intravenous contrast that is required, resulting in an increased risk of nephrotoxicity with consequential renal failure and (iii) increased risk of developing intravascular complications from aggressive wire manipulation, for example vessel wall dissection and/or perforation, distal embolization of blood clots and vessel wall debris and formation of an acute thrombus, all of which can worsen the patient's pre-existing vascular conditions.

Moreover, as more aggressive attempts are made to pass the guidewire across the lesion, there is a tendency to use an increasing number of wires, catheters and adjunct devices, which may result in increased costs and resource utilization.

The nominal sequence of lesion traversal is to attempt manipulation of the bare guidewire across the lesion. If this fails, a catheter can be introduced to provide better torque, manipulation angles as well as to guide the wire tip in the appropriate direction.

The conventional methods for guidewire passage are heavily dependent on 2-dimensional fluoroscopic x-ray imaging that is extra-luminal in nature, in which the blood vessels are visualized externally in 2 planes via x-rays and contrast. Such methods are generally performed under continuous radiation and excessive fluoroscopy time, leading to risks associated with increased radiation exposure.

There is also a significant amount of dependence on hand-eye co-ordination between the operator, the on-screen x-ray images and on tactile feedback during wire/catheter manipulation. Random luck also plays a part in the process. Overall, this results in a series of complex steps requiring focused movements on the operator's part.

In order to improve the passage of the guidewire through the blood vessels, a number of adjuncts have been suggested, such as (i) enhanced fluoroscopic imaging utilising better biplanar or triplanar fluoroscopic machines with improved resolution and magnification, (ii) repeated fluoroscopy imaging at different angles, coupled with more contrast, (iii) use of smaller and lower profile guidewires and catheters, which are costlier, (iv) use of stiffer guidewires, which may have a higher risk of causing vessel wall dissection and perforation and (v) catheters or devices incorporating vessel wall plaque removal or excision systems, vessel sub-intimal wire passage systems, amongst others, which increase cost.

However, the above methods are unsatisfactory in several aspects, e.g. increased radiation exposure, increased contrast use with associated increased risk of renal injury and higher cost. Furthermore, specialized training is required, with very specific usage criteria and variable success rates.

The fact that there are so many adjuncts available suggests that none is clearly advantageous over the others.

Presently, pressure wires or pressure sensors are also used with catheters. However, these sensors provide only pressure data and contact force information, which may not provide sufficient data for the operators to navigate the catheters through the blood vessels. Furthermore, these sensors may be too big for incorporation onto guidewires.

### SUMMARY

According to an embodiment, a micro-sensory tip for use in blood vessels is provided. The micro-sensory tip may include: a force transmission element; at least three force detecting sensors coupled to the force transmission element, each of the at least three force detecting sensors responsive to force applied on the force transmission element, wherein each of the at least three force detecting sensors produces an output representing at least one force component of a three-dimensional Cartesian co-ordinate system, of the force experienced by the force transmission element, such that the outputs of the at least three force detecting sensors can cover the space of the three-dimensional Cartesian co-ordinate system; and an active element arrangement coupled to the at least three force detecting sensors, the active element arrangement configured to process the output from the at least three force detecting sensors.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1A illustrates the passage of a guidewire through a blood vessel.

FIG. 1B shows an expanded perspective view of a guidewire incorporating a tactile sensor at one end of the guidewire, according to one embodiment.

FIG. 1C shows a perspective view of a tactile sensor of an embodiment.

FIG. 2A shows a perspective view of a tactile sensor of an embodiment.

FIG. 2B shows an expanded view of a cantilever of the tactile sensor of the embodiment of FIG. 2A.

FIG. 3 shows an exploded cross sectional view of a micro-sensory tip, according to one embodiment.

FIG. 4 shows a schematic diagram illustrating an application specific integrated circuitry (ASIC), according to one embodiment.

FIG. 5 shows a flow chart illustrating a method of forming a micro-sensory tip, according to various embodiments.

FIGS. 6A to 6J show cross-sectional views of a fabrication process to manufacture a tactile sensor, according to various embodiments.

FIGS. 7A to 7C show cross-sectional views of a process for bonding a polymer ball to a stylus, according to an embodiment.

FIG. 8 is an SEM image showing a side view of a structure with an attached ball, according to various embodiments.

FIG. 9 shows a diagram illustrating vector information displayable on a display unit, according to one embodiment.

FIGS. 10A and 10B show a top view and a cross-sectional view of a measurement set-up to characterize the silicon nanowire of a tactile sensor of an embodiment.

FIGS. 11A and 11B show measurement data of the silicon nanowire of a tactile sensor of the embodiment of FIG. 10.

FIG. 12 shows simulation data illustrating the forces acting on the tactile sensor of one embodiment.

FIGS. 13A and 13B show plots of parameters for the sensors of a tactile sensor according to an embodiment, under different force loadings.

FIG. 14 shows a plot of parameters for the sensors of a tactile sensor according to an embodiment.

FIGS. 15A to 15C show various embodiments of the coupling structures.

FIG. 16 shows sensitivity data for the coupling structures of the embodiments of FIGS. 15A and 15B.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Various embodiments provide a device that may improve the passage of guidewires or catheters through blood vessels and provide contact force information, without or with reduced at least some of the associated disadvantages of the current methods.

Various embodiments may provide a miniaturized, sensitive and robust tri-axial force sensor in a stack assembly with one or more application specific integrated circuits (ASICs) for medical devices. Various embodiments provide a micro-sensory tip for mounting onto the tip of a guidewire that can provide force-related information or data as well as the direction of movement of the guidewire to the operator during the traversal of the guidewire through the body cavity, duct, or vessel of an organism.

Additionally, various embodiments may be used as a tissue characterization tool to provide data on the types of tissues that the guidewire comes into contact with or to characterize the hardness of the vascular vessel walls or calcified tissues.

By providing force-related information and the direction of movement on a display device to the operator, various embodiments may provide force feedback to the operator to navigate the guidewire, thereby minimizing damage to the walls of the blood vessels or tissues.

Various embodiments may also eliminate the need for an imaging system or the use of excessive dosage of radiations and intravenous contrasts for imaging the guidewire during passage through the blood vessels of an organism.

In the context of various embodiments, the term “micro-sensory tip” may mean a miniaturized device including sensors, to be mounted on a tip of a device configured to navigate or traverse through a body cavity, duct, or vessel. The micro-sensory tip may be responsive to forces exerted on the micro-sensory tip and may produce outputs corresponding to the forces.

In the context of various embodiments, the term “micro-sensory tip” may include an assembly of a tactile sensor and an active element arrangement. The assembly may be a stack assembly.

In the context of various embodiments, the term “tactile sensor” may mean a sensor including a force transmission element and a number of force detecting sensors coupled to the force transmission element. In the context of various embodiments, the term “active element arrangement” may mean an arrangement which may include active elements with the ability to electrically control electron flow or current. The arrangement may be in the form of an electronic circuit.

In the context of various embodiments, the term “force transmission element” may mean an element which is responsive to forces exerted on the micro-sensory tip, and may cause deflections of the internal structures of the micro-sensory tip to transmit the forces to the sensors. In various embodiments, the force transmission element may also act as a mechanical stopper, preventing deflections of the internal structures of the micro-sensory tip when a force exceeding a threshold level is exerted on the micro-sensory tip.

The term “force detecting sensors” may mean sensors that are responsive to forces. In the context of various embodiments, the term “responsive” may mean that properties of the sensors may change as a result of the forces. Each force detecting sensor may mean a micro or nano-sized element. In various embodiments, each force detecting sensor may be a piezoresistive nanostructure whose resistance or conductance changes in response to the force experienced. Each force detecting sensor may be formed of polycrystalline silicon or single crystal silicon. Each force detecting sensor may be disposed thereon or therein a cantilever.

In various embodiments, the micro-sensory tip may include a Micro-Electro-Mechanical Systems (MEMS) sensor structure. In the context of various embodiments, the term “electro-mechanical” may mean that the electrical properties of the sensor may change when the sensor is subjected to mechanical forces that alter the shape or actuate the sensor.

The term “coupled” may mean that the force detecting sensors are in communication with the force transmission element such that forces exerted on the force transmission element may be transmitted to the force detecting sensors.

The term “output” may mean information, data or signals generated in response to the forces experienced.

In the context of various embodiments, the term “three-dimensional Cartesian co-ordinate system” may mean a co-ordinate system defined by three axes at right angles to each other, forming a three dimensional space. In various embodiments, a component of a three-dimensional Cartesian co-ordinate system may mean one of the three axes such that the term “one force component” may mean a vector force resolved in the direction of any one of the three axes.

In various embodiments, the active element arrangement may include one or more application specific integrated circuits (ASICs). The application specific integrated circuitry may include a sensor interface and multiplexer coupled to the force detecting sensors to process, amplify, condition and multiplex the signals or outputs from the force detecting sensors. The application specific integrated circuitry may further include a data converter coupled to the sensor interface and multiplexer to receive signals from the sensor interface and multiplexer. The data converter block may include an analog to digital converter to convert the analog signals from the sensor interface and multiplexer into digital data. The application specific integrated circuitry may further include an interface with external reader coupled to the data converter, the interface with external reader receiving and modulating the digital data received from the data converter and transmitting the digital data to an external reader module. The interface with external reader block may also receive decoding command signals from the external reader module. The external reader module may receive the data from the application specific integrated circuitry and communicate with a computer to provide data for display on the computer. The application specific integrated circuitry may further include a clock and controller coupled to synchronise the operations of the sensor interface and multiplexer, the data converter and the interface with external reader.

In various embodiments, a method of forming a micro-sensory tip for use in blood vessels is provided. The method may include providing a force transmission element; coupling at least three force detecting sensors to the force transmission element, each of the at least three force detecting sensors responsive to force applied on the force transmission element, wherein each of the at least three force detecting sensors produces an output representing at least one force component of a three-dimensional Cartesian co-ordinate system, of the force experienced by the force transmission element, such that the outputs of the at least three force detecting sensors can cover the space of the three-dimensional Cartesian co-ordinate system; and coupling an active element arrangement to the at least three force detecting sensors, the active element arrangement configured to process the output from the at least three force detecting sensors.

Various embodiments provide a micro-sensory tip including a tactile force sensor that is sensitive and robust. The micro-sensory tip is designed and packaged to achieve compatibility for mounting at one end or a tip of a guidewire or a catheter. In one embodiment, a micro-sensory tip including tri-axial force sensor is provided at the tip of a guidewire.

In various embodiments, the tactile force sensor may be a Micro-Electro-Mechanical Systems (MEMS) sensor structure, including a force transmission element, in the form of a protruding structure. The force transmission element acts to transmit the force applied on the force transmission element to a number of highly sensitive force detecting sensors coupled to the force transmission element. The force detecting sensors are responsive to the force applied on the force transmission element.

In various embodiments, the force transmission element also acts as a mechanical stopper when a force exceeding a threshold level is exerted on the force transmission element, thereby improving the robustness of the MEMS sensor structure.

In various embodiments, the MEMS sensor structure is assembled together with application specific integrated circuitry (ASIC) to process and multiplex the force-related information from the MEMS sensor structure in order to simplify communications with an external display device wherein an operator can read the force-related information in real time. The MEMS structure and ASIC are assembled in the form of a stack for mounting onto the tip of a guidewire. The stack assembly of the MEMS structure and ASIC may be within the dimensional limit of the guidewire. In various embodiments, the stack assembly may have a minimum width dimension of about 140 μm (140 micrometer), about 200 μm, about 250 μm or about 300 μm.

Various embodiments of the present invention may provide a tactile sensor with the dimensions of about 350 μm±about 5 μm in diameter and having three detecting axes representing three force vectors. The tactile sensor may have a force detecting functional range of about 0 mN to about 25 mN±0.1 mN with 0.2 mN resolution and <1% hysteresis. The tactile sensor may be sufficiently robust to withstand a force of up to about 10 N. In further embodiments, the tactile sensor may have the dimensions of about 150 μm±about 5 μm in diameter, about 200 μm±about 5 μm in diameter, about 250 μm±about 5 μm in diameter or about 300 μm±about 5 μm in diameter. In further embodiments, the tactile sensor may have a force detecting functional range of about 0 mN to about 30 mN, a force detecting functional range of about 0 mN to about 1 N or a force detecting functional range of about 1 N to about 10 N. In further embodiments, the tactile sensor may be sufficiently robust to withstand a force of up to about 2 N, about 5 N or about 8 N. In further embodiments, the tactile sensor may have <0.5% hysteresis, <2% hysteresis, <3% hysteresis or <5% hysteresis. Accordingly, the various embodiments meet the medical requirements on sensor specifications such as: dimensions of about 350 μm±about 5 μm in diameter, being the general diameter of guidewires, three detecting axes, a detecting force range of up to about 20 mN and the ability to withstand a maximum force of up to about 2 N.

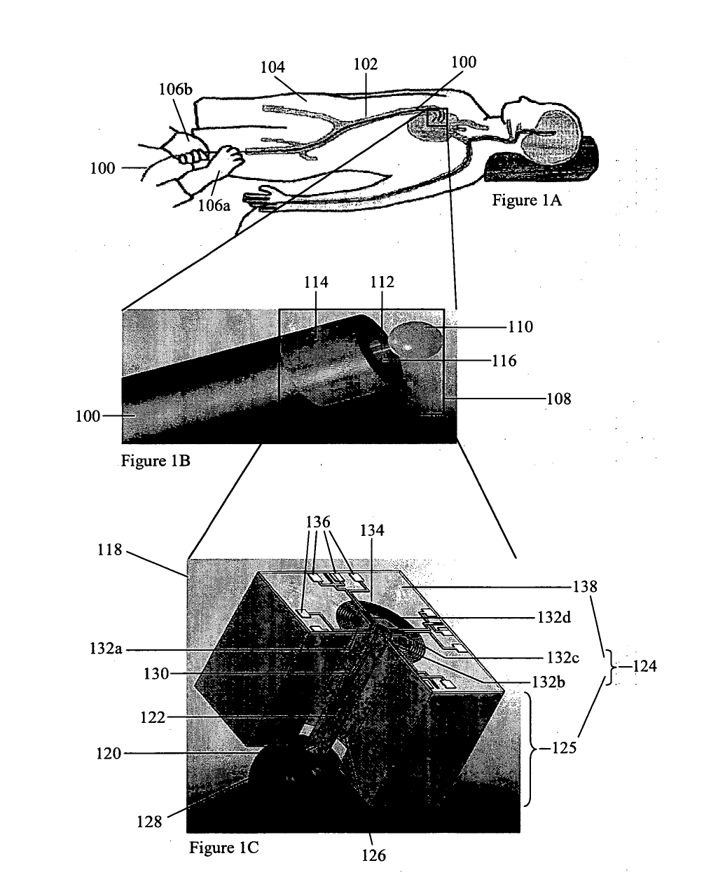

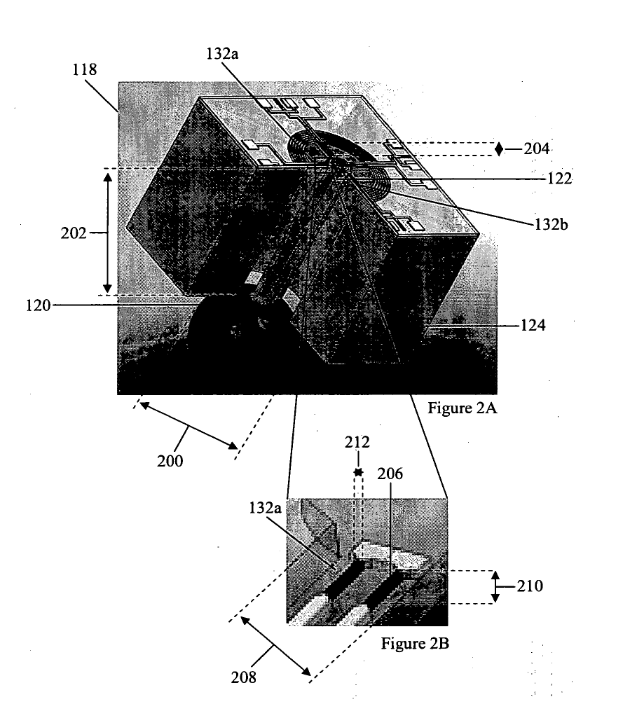

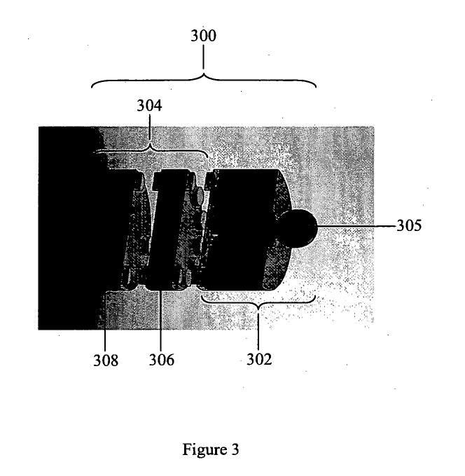

FIG. 1A illustrates the passage of a guidewire 100 through a blood vessel 102. The guidewire 100 may be inserted through an entry point of the organism 104 into the blood vessel 102, in which the guidewire 100 is then guided by an operator using his hands 106a and 106b to navigate the guidewire 100 to traverse inside the blood vessel 102 to a target location within the organism 104.

FIG. 1B shows an expanded perspective view of a guidewire 100, incorporating a tactile sensor 108 at one end of the guidewire 100, according to various embodiments of the present invention. For illustration purposes, an active element arrangement is not shown in FIG. 1B. The tactile sensor 108 includes a force transmission element, in the form of a ball 110, a stylus 112 and a body 114. The body 114 may include a hollow cylindrical structure having a cavity 116 wherein the stylus 112 is located. The perimeter of the cavity 116 is in proximity to the ball 110. The ball 110 is coupled to one end of the stylus 112 passing through the cavity 116, with the other end of the stylus 112 physically coupled to the body 114. The tactile sensor 108 may be a Micro-Electro-Mechanical Systems (MEMS) sensor.

FIG. 1C shows a perspective view of a tactile sensor 118 with a cut-out section to illustrate the stylus 122, in accordance with various embodiments. The tactile sensor 118 may be a Micro-Electro-Mechanical Systems (MEMS) sensor. The tactile sensor 118 may include a force transmission element, in the form of a ball 120, a stylus 122 and a body 124. The body 124 may include a hollow cube having a spacer layer 125 and a surface 138. The spacer layer 125 may include a cavity 126 wherein the stylus 122 is located. The perimeter of the cavity 126 is in proximity to the ball 120. It should be appreciated that the body 124 may take the form and shape of other structures, for example a cuboid.

The ball 120 is coupled to one end 128 of the stylus 122. The stylus 122 passes through the cavity 126, with the other end 130 of the stylus 122 physically coupled to the body 124 via a number of cantilevers 132a, 132b, 132c and 132d.

In the embodiment of FIG. 1C, the ball 120 is coupled to the body 124 via a 4-cross bar structure. The 4-cross bar structure may include the stylus 122 located in the cavity 126 and the four cantilevers 132a, 132b, 132c and 132d, where force detecting sensors (not shown) may be disposed thereon or therein. Accordingly, the spacer layer 125 of the body 124 is located between the ball 120 and the force detecting sensors (not shown). The four cantilevers 132a, 132b, 132c and 132d may be arranged on one common plane. In addition, the four cantilevers 132a, 132b, 132c and 132d may be arranged in a substantially X-shaped arrangement. In various embodiments, the cantilevers 132a, 132b, 132c and 132d may act as the sensing structure where force detecting sensors may be disposed therein or thereon.

The tactile sensor 118 further comprises a series of concentric circles 134, in an outward direction from the cavity 126. The concentric circles 134 function as etch stops during the fabrication process of the tactile sensor 118. The tactile sensor 118 may further include a number of contact pads, for example as represented by 136. The cantilevers 132a, 132b, 132c and 132d, the concentric circles 134 and the contact pads 136 are disposed on the surface 138. The contact pads 136 are provided for electrical communication with circuitry or an active element arrangement that may be positioned adjacent to the surface 138 in a stack assembly.

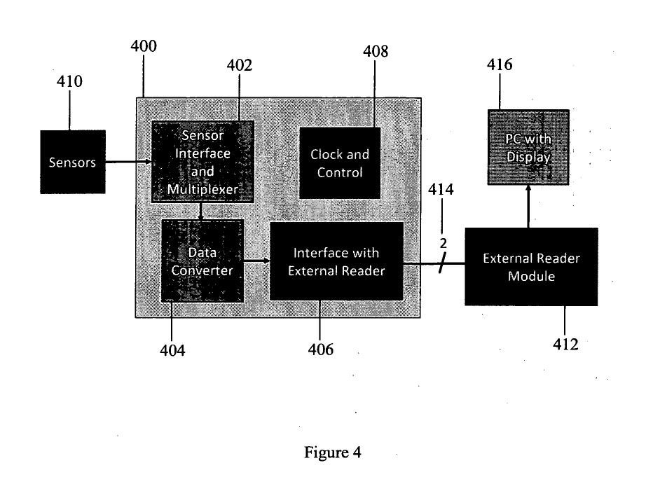

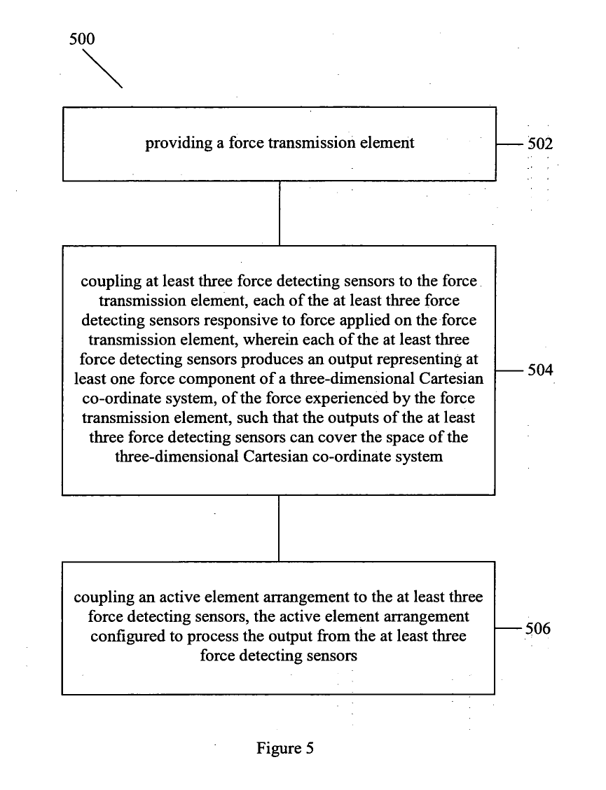

FIG. 2A, which shows a similar perspective view of the tactile sensor 118 of the embodiment illustrated in FIG. 1C, is shown to provide representations of the dimensions of the tactile sensor 118. The ball 120 is coupled to the body 124 via a 4-cross bar structure including the stylus 122 and the four cantilevers, for example 132a and 132b represented for two of the four cantilevers.

The tactile sensor 118 of various embodiments may have the following parameters. The diameter 200 of the ball 120 may be approximately 350 μm. The stylus length 202 of the stylus 122 may be 450 μm. The cantilever length 204 of each of the cantilevers 132a, 132b may be 100 μm.

FIG. 2B shows an expanded view of the cantilever 132a of the tactile sensor 118 of the embodiment of FIG. 2A. It should be appreciated that while FIG. 2B illustrates the cantilever 132a, FIG. 2B is also representative of the other cantilevers, for example the cantilever 132b.

The cantilever 132a may include force detecting sensors, for example as represented by 206, disposed thereon. Accordingly, the cantilever 132a may act as a sensing structure where the force detecting sensors 206 may be disposed thereon. The force detecting sensors 206 are generally nanosized, thereby increasing the sensitivity of the tactile sensor 118. The force detecting sensors may be piezoresistive sensors and change their resistance in response to the force experienced. In various embodiments, the force detecting sensors 206 may include nanowires, e.g. silicon (Si) nanowires. The (e.g. silicon) nanowires may be deformable. The (e.g. silicon) nanowires may be made of polycrystalline silicon or single crystal silicon.

In one embodiment, two force detecting sensors 206 are disposed on the cantilever 132a. The two force detecting sensors 206 disposed on the cantilever 132a may provide differential sensing, thereby providing enhanced sensitivity. In an alternative embodiment, one force detecting sensor 206 may be disposed on the cantilever 132a.

The force detecting sensors 206 on the cantilever 132a are coupled to the force transmission element, in the form of the ball 120. Similarly, force detecting sensors disposed on the cantilever 132b and the remaining cantilevers are also coupled to the force transmission element.

In various embodiments, (e.g. silicon) nanowires are used due to their superior electrical properties, thereby enabling small form factor sensor designs.

Referring to FIG. 2B, in terms of dimensions, the cantilever width 208 of the cantilever 132a may be 30 μm. Using (e.g. silicon) nanowires as the example for the force detecting sensors 206, the length 210 of the sensor 206 may be approximately 1 μm while the diameter 212 of the sensor 206 may be approximately 90 nm.

In various embodiments, it should be appreciated that other materials, for example semiconductor materials such as silicon-germanium (SiGe), germanium (Ge) or gallium arsenide (GaAs), which may exhibit piezoresistive effects, may be used for the force detecting sensors. In various embodiments, piezoresistive materials such as diamond, silicon carbide (SiC) or carbon nanotubes, may be used for the force detecting sensors. In further embodiments, the force detecting sensors may comprise metals. Further, it should be appreciated that the force detecting sensors may take the form of other deformable structures, such as a nanotube.

In further embodiments, the force detecting sensors 206 may include a metal-oxide-semiconductor field-effect transistor (MOSFET) formed therein. The MOSFET may be responsive to force or stress.

It should be appreciated that the ball diameter 200, the stylus length 202, the cantilever length 204 and the cantilever width 208 may have different dimensions, depending on the structure and application of the tactile sensor 118.

In various embodiments of the present invention, the tactile sensor 108, 118 may be assembled together with an active element arrangement positioned adjacent to the tactile sensor 108, 118, to form a micro-sensory tip which can be mounted onto the tip of a guidewire or a catheter. The active element arrangement is in electrical communication with the tactile sensor 108, 118 to process the force-related information or signal from the tactile sensor 108, 118.

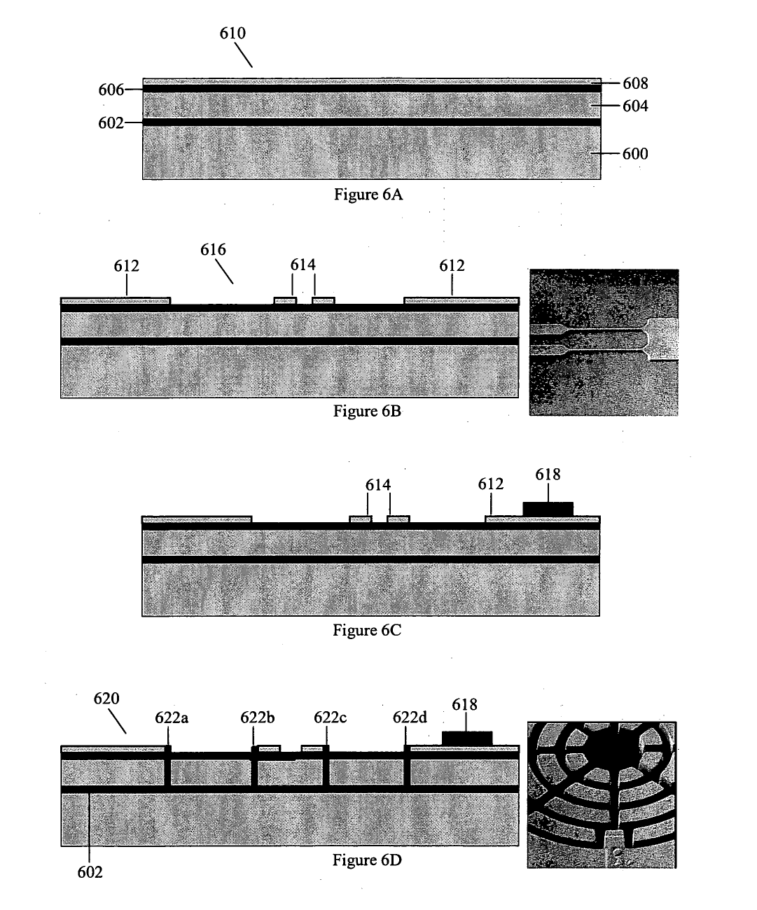

FIG. 3 shows an exploded cross sectional view of a micro-sensory tip 300, according to one embodiment. The micro-sensory tip 300 may include a tactile sensor 302 and an active element arrangement 304. The tactile sensor 302 may include a force transmission element 305. The active element arrangement 304 may include a first application specific integrated circuit (ASIC) 306 and a second application specific integrated circuit (ASIC) 308. The tactile sensor 302 may be a Micro-Electro-Mechanical Systems (MEMS) sensor.

The tactile sensor 302 is assembled together with the active element arrangement 304 including the application specific integrated circuits (ASICs) 306, 308, in the form of a stack to form the micro-sensory tip 300. In various embodiments, the stack assembly of the tactile sensor 302 and the application specific integrated circuits (ASICs) 306, 308 are within the dimensional limit of the guidewire for mounting onto the tip of the guidewire.

The micro-sensory tip 300 may be packaged in a 3D stack wherein the tactile sensor 302 are in electrical communication with the application specific integrated circuits (ASICs) 306, 308 based on through-silicon via (TSV) technology. Through-silicon via (TSV) technology is understood in the art and will not be described here. In alternative embodiments, the application specific integrated circuits (ASICs) 306, 308 may be assembled together with the tactile sensor 302 and coupled to the force detecting sensors (not shown) via flip chip or wire bonding.

The micro-sensory tip 300 may be made of biocompatible packaging. The micro-sensory tip 300 may provide a human body environment interface as the micro-sensory tip 300 may directly make contact with, for example, the blood vessel lumen of an organism.

In an alternative embodiment, the active element arrangement 304 may comprise only one application specific integrated circuit (ASIC) 306 assembled with the tactile sensor 302 to form the micro-sensory tip 300. In a further alternative embodiment, the active element arrangement 304 may comprise more than two application specific integrated circuits (ASICs) assembled with the tactile sensor 302 to form the micro-sensory tip 300.

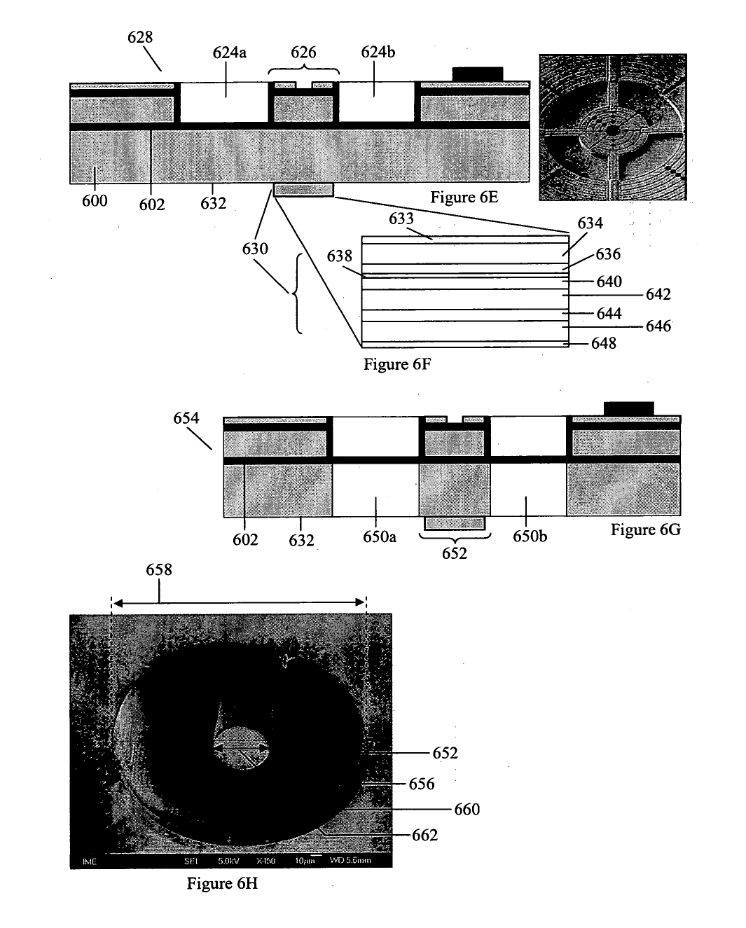

FIG. 4 shows a schematic diagram illustrating an application specific integrated circuit (ASIC) 400 of an active element arrangement. In various embodiments, the application specific integrated circuit (ASIC) 400 may include a number of functional blocks such as a sensor interface and multiplexer block 402, a data converter block 404, an interface with external reader block 406 and a clock and control block 408.

The sensor interface and multiplexer block 402 is coupled to the force detecting sensors, represented by the sensors block 410, to process, amplify, condition and multiplex the signals or outputs from the force detecting sensors 410.

The data converter block 404 is coupled to the sensor interface and multiplexer block 402 to receive signals from the sensor interface and multiplexer block 402. The data converter block 404 may include an analog to digital converter to convert the analog signals from the sensor interface and multiplexer block 402 into digital data.

The interface with external reader block 406 is coupled to the data converter block 404 to receive and modulate the digital data received from the data converter block 404. The interface with external reader block 406 then transmits the digital data to the external reader module 412 via a 2-wire interface 414. The interface with external reader block 406 also receives decoding command signals from the external reader module 412 via the 2-wire interface 414.

The external reader module 412 receives the data from the application specific integrated circuit (ASIC) 400 and communicates with a computer, represented by the PC with display block 416, to provide data for display of the force-related information on the computer.

A clock device, represented by the clock and control block 408, may be coupled to the sensor interface and multiplexer block 402, the data converter block 404 and the interface with external reader block 406, in order to supply clock and control signals to the various functional blocks, thereby synchronizing the operations of the functional blocks within the application specific integrated circuit 400.

It should be appreciated that the coupling between the functional blocks within the application specific integrated circuitry (ASIC) 400 may be by electrical connections, for example electrical wires.

In various embodiments, the application specific integrated circuitry (ASIC) 400 may have dimensions of about 350 μm×about 350 μm or smaller.

Referring to FIGS. 3 and 4, the micro-sensory tip 300 detects the force acting on the force transmission element 305 of the tactile sensor 302 and transmits the force to the force detecting sensors 410 while the active element arrangement 304 coupled to the force detecting sensors 410 is configured to process the output from the force detecting sensors 410 to produce force-related information for communications and transmission to an external display device 416. An operator navigating a guidewire with the micro-sensory tip 300 mounted on the guidewire can read the force-related information in real time on the display device 416.

In various embodiments, at least three force detecting sensors coupled to the force transmission element are provided, such that each of the at least three force detecting sensors produces an output representing at least one force component of a three-dimensional Cartesian co-ordinate system, of the force experienced by the force transmission element, such that the outputs of the at least three force detecting sensors can cover the space of the three-dimensional Cartesian co-ordinate system. The active element arrangement coupled to the at least three force detecting sensors then processes the output from the at least three force detecting sensors.

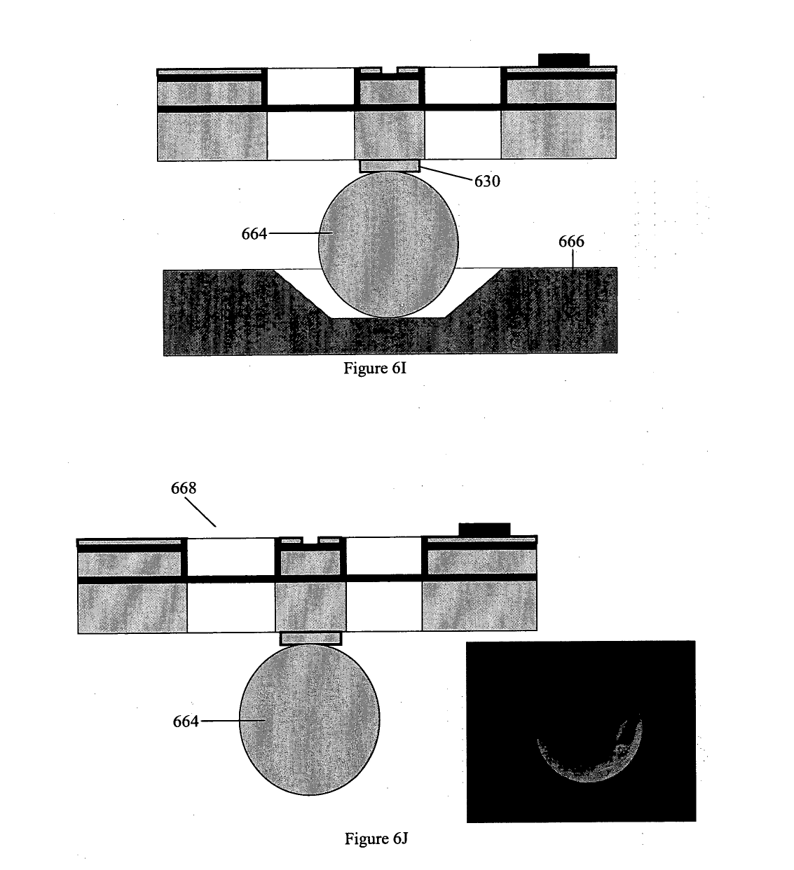

FIG. 5 shows a flow chart 500 illustrating a method of forming a micro-sensory tip, according to various embodiments.

At 502, a force transmission element according to various embodiments is provided.

At 504, at least three force detecting sensors are coupled to the force transmission element, each of the at least three force detecting sensors responsive to force applied on the force transmission element, wherein each of the at least three force detecting sensors produces an output representing at least one force component of a three-dimensional Cartesian co-ordinate system, of the force experienced by the force transmission element, such that the outputs of the at least three force detecting sensors can cover the space of the three-dimensional Cartesian co-ordinate system.

At 506, an active element arrangement is coupled to the at least three force detecting sensors, the active element arrangement configured to process the output from the at least three force detecting sensors.

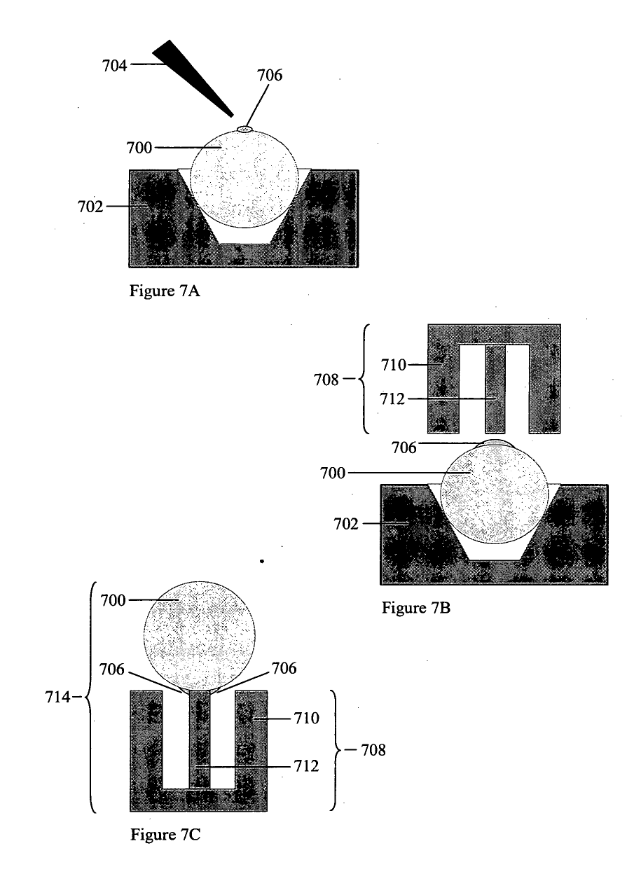

FIGS. 6A to 6J show cross-sectional views of a fabrication process to manufacture a tactile sensor (for example the tactile sensor 108 of FIG. 1B or tactile sensor 118 of FIGS. 1C and 2A), according to various embodiments.

In FIG. 6A, a substrate 600 may be provided. The substrate 600 may be silicon. A buried oxide layer 602, for example a silicon dioxide layer, is formed on the substrate 600, followed by a semiconductor layer 604. The semiconductor layer 604 may be a layer of polycrystalline silicon or single crystal silicon. A second buried oxide layer 606, for example a silicon dioxide layer, is then formed on the semiconductor layer 604, followed by a semiconductor layer 608. The semiconductor layer 608 may be a layer of single crystal silicon. The semiconductor layer 608 is used for the fabrication of the nanosized sensing elements acting as the force detecting sensors. Accordingly, the tactile sensor may be fabricated using a double silicon on insulator (SOI) structure 610, as shown in the embodiment of FIG. 6A.

In an alternative embodiment, the tactile sensor may be fabricated using a silicon on insulator (SOI) structure, comprising the substrate 600, the buried oxide layer 602 and the semiconductor layer 604. The buried oxide layer 602 may be a silicon dioxide layer and the semiconductor layer 604 may be a layer of polycrystalline silicon or single crystal silicon. The semiconductor layer 604 is used for the fabrication of the nanosized sensing elements acting as the force detecting sensors.

In a further embodiment, the tactile sensor may be fabricated using a bulk silicon wafer, in which the nanosized sensing elements acting as the force detecting sensors may be fabricated thereon or therein. The bulk silicon wafer may be polycrystalline silicon.

In various embodiments, the structure or wafer for the fabrication of the tactile sensor may be about 0.5 μm to about 100 μm, depending on the applications.

Using the embodiment shown in FIG. 6A, an implantation process using the dopant boron (B) is performed on the structure 610 in order to make the semiconductor layer 608 conductive and to tune the piezoresistive properties. The semiconductor layer 608 may be selectively patterned or etched, resulting in the etched semiconductor layer 612 shown in FIG. 6B, that may be used to define nanowire structures 614. An oxidation process is then carried out on the structure 616 to create a layer of dielectric on the surface of the nanowire structures 614. A scanning electron microscope (SEM) image showing a top view of the structure 616 formed is shown on the right of FIG. 6B.

A second implantation process using a relatively higher concentration of the dopant boron (B) than the first implantation process, is carried out on the structure 616, except on the nanowire structures 614. The second implantation process is performed to form low resistance ohmic contact. Next, a pre-metal dielectric layer is deposited and patterned to open a contact to one portion of the etched semiconductor layer 612. Subsequently, a metal layer is deposited and patterned to form a contact pad 618, as shown in FIG. 6C. The contact pad 618 may be made of gold.

Stiffeners are then formed to function as etch stops to define the cantilever anchor boundary. Openings are created by etching until the buried oxide layer 602, where a stiffening material is provided to fill the openings and to form a number of stiffeners 622a, 622b, 622c and 622d, as shown in FIG. 6D. For illustration purposes, four stiffeners 622a, 622b, 622c and 622d are shown in FIG. 6D. However, it should be appreciated that any number of stiffeners may be formed. The stiffening material may be an oxide such as e.g. silicon dioxide. An SEM image showing a top view of the structure 620 formed is shown on the right of FIG. 6D.

Selective patterning or etching is then carried out on the structure 620 of FIG. 6D until the buried oxide layer 602 to create etched segments 624a, 624b, to form and define the cantilever, for example 626, as shown in FIG. 6E. Subsequently, the substrate 600 may be thinned to reduce the thickness of the substrate 600. An SEM image showing a top view of the structure 628 formed is shown on the right of FIG. 6E.

In various embodiments, an under bump metallization (UBM) 630 is formed on a substantially central location on the surface 632 of the substrate 600, as shown in FIG. 6E. In alternative embodiments, a gold layer may be formed instead of an under bump metallization (UBM).

The under bump metallization (UBM) 630 may have a structure as shown in FIG. 6F. The layer 633 may be a titanium (Ti) layer with a thickness of approximately 50 nm. The layer 634 may be a copper (Cu) layer with a thickness of approximately 2 μm. The layer 636 may be a nickel (Ni) layer with a thickness of approximately 100 nm. The layer 638 may be a gold (Au) layer with a thickness of approximately 10 nm. The layer 640 may be an indium (In) layer with a thickness of approximately 1.5 μm. The layer 642 may be a tin (Sn) layer with a thickness of approximately 2 μm. The layer 644 may be an indium (In) layer with a thickness of approximately 1.5 μm. The layer. 646 may be a tin (Sn) layer with a thickness of approximately 2 μm. The layer 648 may be a gold (Au) layer with a thickness of approximately 30 nm.

Etching or deep reactive-ion etching (DRIE) is then carried out on the structure 628 of FIG. 6E from the surface 632 until the buried oxide layer 602 to create etched segments 650a, 650b, as shown in FIG. 6G, in order to define the stylus 652. Although not clearly shown in FIG. 6E, the etched segments 650a, 650b are part of a continuous etched cavity surrounding the stylus 652.

FIG. 6H shows an SEM image showing a bottom view of the structure 654 formed to illustrate the stylus 652 and the cavity 656 around the stylus 652, as a result of the deep reactive-ion etching process. The diameter 658 of the perimeter 660 of the cavity 656 is estimated to be about 209 μm. The diameter 662 of the stylus 652 is estimated to be about 47.5 μm.

A ball 664 on a silicon holder 666 is brought in contact with the UBM 630 for attachment onto the UBM 630, as shown in FIG. 6L The ball 664 may be a solder ball. In various embodiments, the ball may be attached to the UBM 630 using a ball placement method, a ball drop method or bonding.

FIG. 6J shows the final structure 668 of the tactile sensor. An SEM image showing the ball 664 is shown on the right of FIG. 6J.

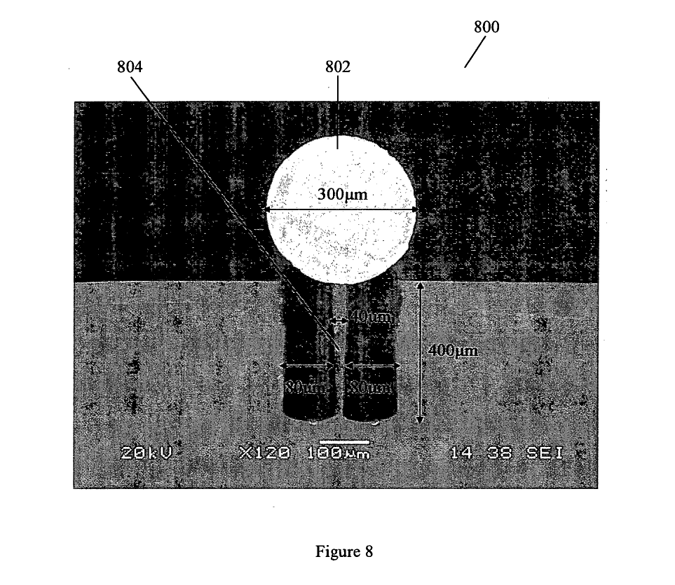

In alternative embodiments, a polymer ball may be used. FIGS. 7A to 7C show cross-sectional views of a process for bonding a polymer ball 700 to a stylus 712, according to an embodiment.

As shown in FIG. 7A, the polymer ball 700 is placed on a silicon holder 702. A dispenser 704 is used to dispense epoxy 706 on the top of the polymer ball 700.

Then, using a flip chip bonder, a structure 708, comprising a body 710 and a stylus 712 as part of a tactile sensor is aligned with the epoxy 706 and the polymer ball 700, as shown in FIG. 7B. The structure 708 is brought into contact with the epoxy 706 for bonding.

A baking process is then carried out to cure the epoxy 706. FIG. 7C shows the final structure 714, with the polymer ball 700 attached to the stylus 712 via epoxy 706 bonding.

In various embodiments, the polymer ball 700 may be biocompatible.

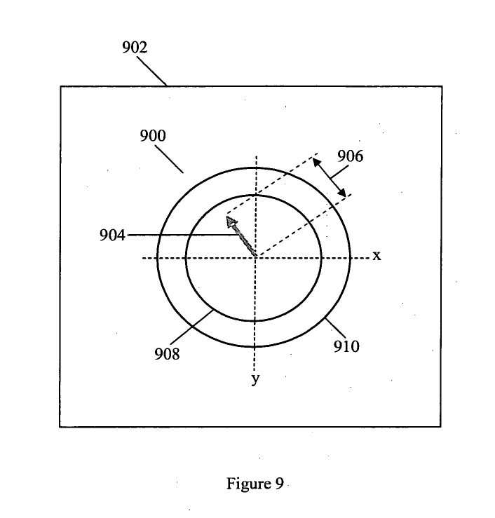

FIG. 8 is an SEM image showing a side view of the structure 800 with a ball 802 attached to the stylus 804 of the structure 800, according to various embodiments.

In various embodiments, the ball (664, 700, 802) acts as a force transmission element. Additionally, the ball (664, 700, 802) acts as a mechanical stopper. In further embodiments, the ball (664, 700, 802) may act as a seismic mass with off-set from the sensing plane and hence may act as a tri-axial accelerometer.

In alternative embodiments, other structures in the form of a cuboid, a prism, a cylinder and a pyramid may be used instead of the ball (664, 700, 802).

In general, any structures with dimensions relatively larger than the perimeter 660 of the cavity 656 may be used.

After the final structure of the tactile sensor is fabricated, it may be packaged together with one or more application specific integrated circuits (ASICs) to form a micro-sensory tip, such as the embodiment shown in FIG. 3.

Various embodiments of the micro-sensory tip can be implemented on guidewires, catheters and other medical devices where tri-axial force data may be required. The operation of the micro-sensory tip as mounted on a guidewire will now be described as follows, by way of examples and not limitations.

A micro-sensory tip comprising a stack assembly of a tactile sensor in communication with an active element arrangement, according to various embodiments, is mounted on a distal tip of a guidewire.

The tactile sensor may include a force transmission element, in the form of a ball coupled to a stylus, which in turn is coupled to the body of the tactile sensor. The ball also acts as a mechanical stopper. At least three force detecting sensors are coupled to the force transmission element, which are responsive to a force applied on the force transmission element such that each of the at least three force detecting sensors produces an output representing at least one force component of a three-dimensional Cartesian co-ordinate system, of the force experienced by the force transmission element. Accordingly, the outputs of the at least three force detecting sensors can cover the space of the three-dimensional Cartesian co-ordinate system. The tactile sensor may be of the embodiments shown in FIGS. 1B, 1C, 2A and 2B.

The active element arrangement comprises an application specific integrated circuit (ASIC) and may be of the embodiments shown in FIGS. 3 and 4.

By way of surgical procedures, the guidewire with the micro-sensory tip is inserted into the blood vessels of an organism. An operator holds the proximal end of the guidewire with his hands and navigates the guidewire through the network of blood vessels within the organism to a particular target location.

During passage of the guidewire with the mounted micro-sensory tip through the network of blood vessels, the micro-sensory tip may come into contact with walls of the blood vessels or tissues. When the micro-sensory tip contacts the wall of a blood vessel, a force is experienced by the ball of the tactile sensor that pushes the ball towards the body of the tactile sensor. The force is transmitted from the ball, through the stylus the ball is coupled to, to the force detecting sensors which may deform as a result of the force.

The force detecting sensors may be piezoresistive sensors, which change their resistance or conductance in response to the force experienced. Such a change causes each of the force detecting sensors to produce an output representing at least one force component of a three-dimensional Cartesian co-ordinate system such that the outputs of the force detecting sensors can cover the space of the three-dimensional Cartesian co-ordinate system.

Subsequently, the application specific integrated circuit (ASIC) coupled to the force detecting sensors processes the outputs from the force detecting sensors and produces an output containing vector information of the force experienced by the ball.

In one embodiment, the application specific integrated circuit (ASIC) may provide the output to a display unit so that the vector information may be displayed as a symbol on two-dimensional Cartesian co-ordinate axes.

In another embodiment, the application specific integrated circuit (ASIC) may provide the output to a display unit so that the vector information may be displayed as a symbol on three-dimensional Cartesian co-ordinate axes.

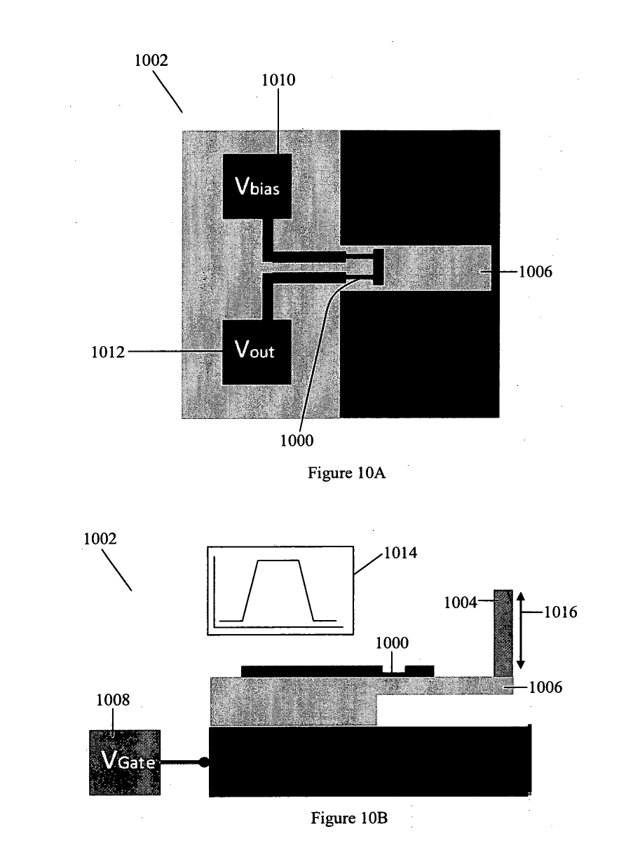

FIG. 9 shows a diagram 900 illustrating vector information displayable on a display unit 902, according to one embodiment. In this embodiment, vector information displayable as a symbol on two-dimensional Cartesian co-ordinate axes is shown. The vector information may be shown in the form of an arrow 904 on x-y Cartesian co-ordinate axes. The direction that the arrow 904 points provides information on the direction of movement of the guidewire while the length 906 of the arrow 904 provides information on the amplitude of the force experienced by the micro-sensory tip and the guidewire. Accordingly, the arrow 904 is a force vector.

The circle 908 may be displayed to indicate a safe margin for the contact force experienced. An arrow 904 with a length 906 entirely within the perimeter of the circle 908 indicates that the contact force experienced by the micro-sensory tip is within a safe margin level. However, should the length 906 exceed the perimeter of the circle 908, the contact force experienced is beyond the safe margin level. For example, this may occur when the micro-sensory tip makes contact with the wall of a blood vessel. Accordingly, the operator of the guidewire can respond by, for example, stopping the passage of the guidewire or changing the direction of the guidewire. In various embodiments, the circle 910 may be displayed to indicate a boundary or a limit for the force detecting functional range of the sensor.

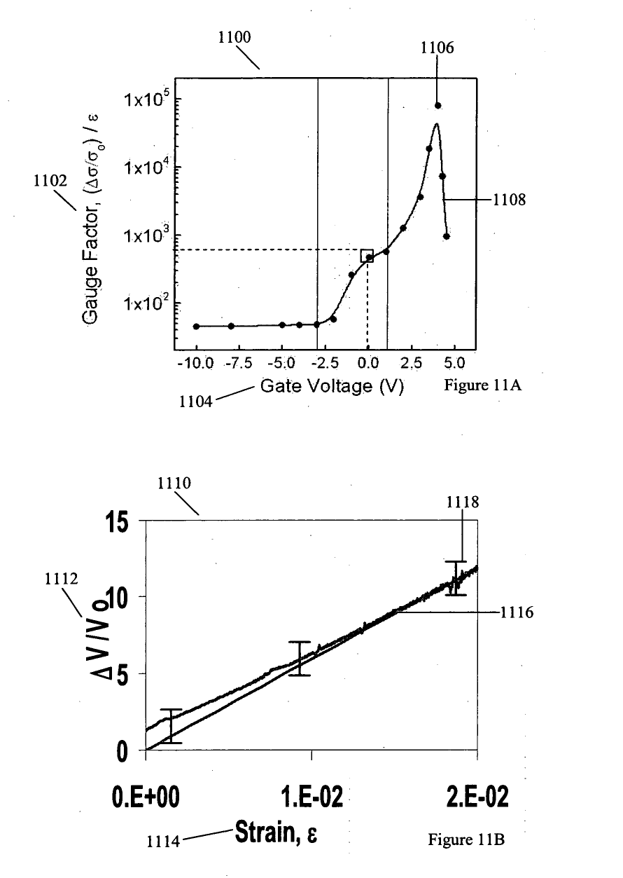

FIGS. 10A and 10B show a top view and a cross-sectional view of a measurement set-up to characterize the silicon nanowire 1000 of a tactile sensor 1002, according to one embodiment. For illustration purposes, only one cantilever 1006 is shown. Also shown as part of the tactile sensor 1002 of FIGS. 10A and 10B are a stylus 1004 and a number of electrical contact pads, such as the gate voltage 1008, bias voltage 1010 and output voltage 1012.

The silicon nanowire 1000 may have a size of approximately 0.12 mm² (0.12 square millimeter). The piezoresistance of the silicon nanowire 1000, defined as the ratio of change in conductance over applied strain, may be 300. In contrast, a conventional MEMS sensor may have a size of 1 mm² and a piezoresistance value of 200. The silicon nanowire 1000 may be CMOS compatible.

For the characterization measurement, the stylus 1004 may be subjected to a cyclic load or cyclic force, where the inset graph 1014 of FIG. 10B represents one cycle of the load applied to the stylus 1004. This causes the stylus 1004 to move up and down as represented by the arrow 1016. As a result of the movement of the stylus 1004, the cantilever 1006 may be deflected, thereby causing a change in the resistance of the silicon nanowire 1000 and producing an output signal corresponding to the force exerted.

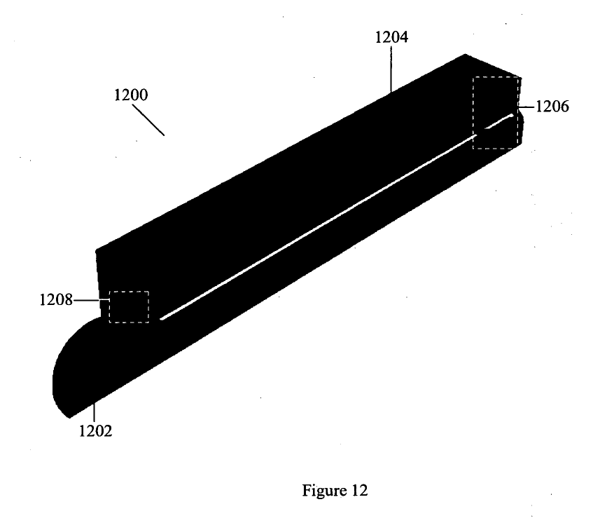

FIG. 11A shows a graph 1100 of the gauge factor 1102, also known as piezoresistivity, against the gate voltage 1104, for the silicon nanowire. Measurement data, for example as represented by the circle 1106, are plotted for various gate voltages 1104. A line fit 1108 is provided for the data 1106.

FIG. 11B shows a graph 1110 of the ratio of change in voltage over the initial voltage 1112 against the applied strain 1114 at the gauge factor value of 600 and zero gate voltage. The data shows a hysteresis of less than 1% and a first order polynomial linearity fit 1116. The symbols ‘I’, represented for example as 1118, represent possible errors in the measurement data. Fatigue measurement indicates that the silicon nanowire remain operational for more than 10⁶ cycles (data not shown).

In various embodiments, the ball of the tactile sensor also acts as a mechanical stopper. The tactile sensor may be configured to operate within the functional range of force of 0 to 25 mN. The upper limit of 25 mN may be set as the force threshold level. In the event that a relatively larger force exceeding this threshold level is experienced by the ball, the ball may be pushed against the body of the tactile sensor. However, as the ball has a relatively larger diameter than the diameter of the perimeter of the cavity through which the stylus coupled to the ball passes through, the ball comes into contact with the perimeter of the cavity and is stopped from being pushed deeper into the body of the tactile sensor. This prevents deformation or damage of the tactile sensor with its associated internal structures and the overall micro-sensory tip, thereby increasing the robustness of the micro-sensory tip.

Simulation on the performance and the forces experienced by the tactile sensor, according to embodiments, was performed using finite element method. This model takes into consideration complex contact mechanics. Finite element method is understood in the art and will not be described here.

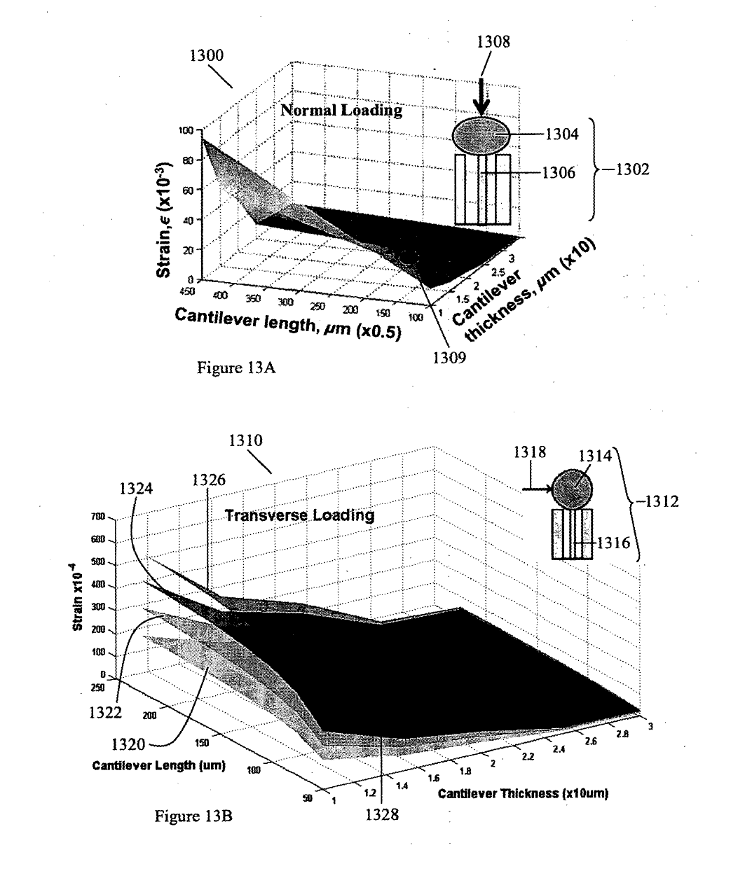

FIG. 12 shows simulation data illustrating the forces acting on the tactile sensor 1200 of one embodiment. For illustration purposes, only a partial cross sectional view of the tactile sensor 1200 is shown.

When the force transmission element, in the form of a ball 1202 (or alternatively called a spatula due to its shape as it appears in FIG. 12), experiences a force, the ball 1202 is pushed towards the body 1204 of the tactile sensor 1200 and the force is transmitted to the region 1206 where the cantilevers with the nanosized force detecting sensors (not shown) are located. Based on the deformation patterns of the cantilevers, the strain may vary and the nanosized force detecting sensors may respond to the strain exerted by changing the electrical resistance (or conductance). Subsequently, the application specific integrated circuit (ASIC) coupled to the nanosized force detecting sensors may respond to the changes and produce an output.

When the ball 1202 experiences a relatively larger force exceeding a threshold level, the ball 1202 is pushed towards the body 1204 and as the ball 1202 also acts as a mechanical stopper, the ball 1202 comes into contact with the perimeter of the cavity of the body 1204, preventing further movement of the ball 1202. In this case, a force is also experienced in the region 1208, in addition to the force experienced in the region 1206.

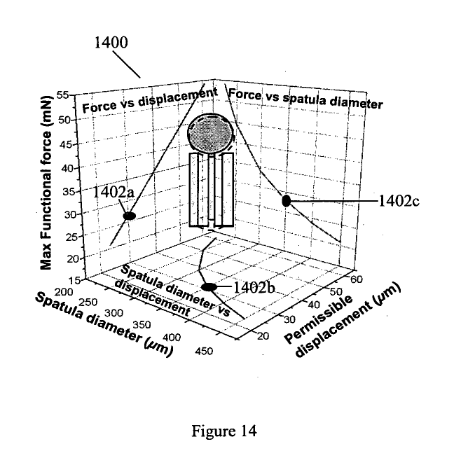

FIGS. 13A and 13B show the relationships of the design parameters for the tactile sensor, under normal loading and transverse loading, respectively. FIG. 13A shows a plot 1300 of parameters for the sensors of a tactile sensor 1302 according to an embodiment, under normal loading. Under normal loading, a force is applied to the top of the ball 1304 in a direction, as represented by the arrow 1308, substantially parallel to the longitudinal axis of the stylus 1306.

FIG. 13B shows a plot 1310 of parameters for the sensors of a tactile sensor 1312 according to an embodiment, under transverse loading for styluses with different lengths. Under transverse loading, a force is applied from the side of the ball 1314 in a direction, as represented by the arrow 1318, substantially perpendicular to the longitudinal axis of the stylus 1316. FIG. 13B shows the results for a tactile sensor 1312 having a stylus 1316 with a length of about 200 μm 1320, about 300 μm 1322, about 400 μm 1324 and about 500 μm 1326. The circles 1309 and 1328 of FIGS. 13A and 13B respectively, provide an example of a possible set of design parameters for the tactile sensor, which may fulfil the requirements of various applications in which the tactile sensor may be used for.

The plot 1300 of FIG. 13A is generated based on an embodiment of a tactile sensor having the following structural parameters: 4 cross cantilevers, a stylus length of approximately 450 μm and a ball diameter of approximately 350 μm. The plot 1310 of FIG. 13B is generated based on an embodiment of a tactile sensor having the following structural parameters: 4 cross cantilevers and a ball diameter of approximately 350 μm.

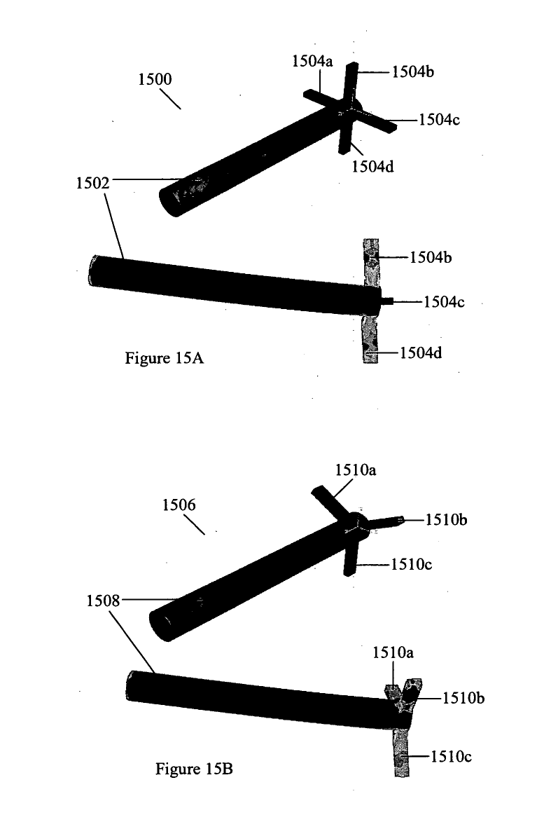

FIG. 14 shows a plot 1400 of parameters for the sensors of a tactile sensor according to an embodiment, for balls (or spatulas) with different diameters. FIG. 14 shows that for a ball or a spatula with a diameter of approximately 350 μm, a 25 mN force applied to the ball causes the stylus to displace by approximately 30 μm. The circles 1402a, 1402b and 1402c provide an example of a possible set of design parameters for the tactile sensor, which may fulfil the requirements of various applications in which the tactile sensor may be used for.

The plot 1400 is generated based on an embodiment of a tactile sensor having the following structural parameters: 4 cross cantilevers, each cantilever having a length of approximately 100 μm and a width of approximately 30 μm and a stylus length of approximately 450 μm.

In various embodiments, the structural parameters and the electrical performance (such as the gauge factor of the silicon nanowires) are linked and accordingly, these factors should be considered to design a tactile sensor that can perform at an optimum level.

In various embodiments, the dimension of the ball determines the functional range of the tactile sensor. A relatively larger diameter provides a relatively smaller functional range. In various embodiments, a 350 μm diameter ball provides a functional range of up to 25 mN.

In various embodiments, the length of the stylus has an effect on the transverse loading conditions. A relatively longer stylus may provide enhanced detection sensitivity for the force encountered under transverse loading. A relatively longer stylus may be formed by providing a deeper etching process during the fabrication process.

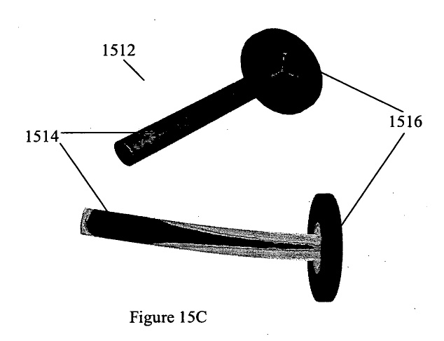

In various embodiments of the present invention, coupling structures are provided in the tactile sensors to couple the force transmission element to the body of the tactile sensor and the force detecting sensors. FIG. 15A shows an embodiment of a 4-cross bar structure 1500 having a stylus 1502 and four cantilevers 1504a, 1504b, 1504c and 1504d. Four force detecting sensors may be provided, with each force detecting sensor disposed on each of the cantilevers 1504a, 1504b, 1504c and 1504d. The force detecting sensors may be arranged on one common plane. The force detecting sensors may be arranged in a substantially X-shaped arrangement. Accordingly, the angular displacement between adjacent force detecting sensors may be provided such that the force detecting sensors form a substantially X-shaped arrangement. The force detecting sensors may be piezoresistive sensors. Simulation data illustrating the forces acting on the stylus 1502 and the cantilevers 1504a, 1504b, 1504c and 1504d is shown at the bottom of FIG. 15A.

Alternative embodiments in place of the 4-cross bar structure 1500 may be provided in order to reduce the number of inputs and outputs (I/Os) and to improve the sensitivity of the tactile sensor.

FIG. 15B shows an embodiment of a 3-cross bar structure 1506 including a stylus 1508 and three cantilevers 1510a, 1510b and 1510c. Three force detecting sensors may be provided, with each force detecting sensor disposed on each of the three cantilevers 1510a, 1510b and 1510c. The force detecting sensors may be arranged on one common plane. The force detecting sensors may be arranged in a substantially Y-shaped arrangement. Accordingly, the angular displacement between adjacent force detecting sensors may be provided such that the force detecting sensors form a substantially Y-shaped arrangement. The force detecting sensors may be piezoresistive sensors. Simulation data illustrating the forces acting on the stylus 1508 and the three cantilevers 1510a, 1510b and 1510c is shown at the bottom of FIG. 15B.

FIG. 15C shows an embodiment of a circular diaphragm structure 1512 comprising a stylus 1514 and a circular diaphragm 1516. Four force detecting sensors may be disposed on the circular diaphragm 1516. The force detecting sensors may be arranged on one common plane. The force detecting sensors may be piezoresistive sensors. Simulation data illustrating the forces acting on the stylus 1514 and the circular diaphragm 1516 is shown at the bottom of FIG. 15C.

In various embodiments of FIGS. 15A to 15C, at least three force detecting sensors coupled to the force transmission element may be provided, with each of the at least three force detecting sensors producing an output representing at least one force component of a three-dimensional Cartesian co-ordinate system, of the force experienced by the force transmission element, such that the outputs of the at least three force detecting sensors can cover the space of the three-dimensional Cartesian co-ordinate system.

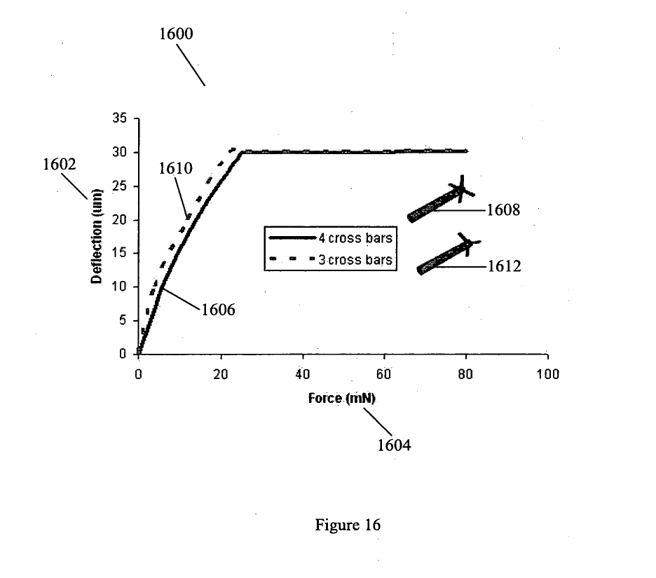

FIG. 16 shows the sensitivity data for the coupling structures of the embodiments of FIGS. 15A and 15B, in a graph 1600 of deflection 1602 against force 1604. The solid line 1606 shows the measurement data for a 4-cross bar structure 1608 while the dashed line 1610 shows the measurement data for a 3-cross bar structure 1612.

FIG. 16 shows that for a given force in the range of 0 to 25 mN, the 3-cross bar structure 1612 experiences a relatively larger deflection 1602 as shown by the dashed line 1610, compared to the 4-cross bar structure 1608. This illustrates that the 3-cross bar structure 1612 has a relatively higher sensitivity, more flexibility and higher efficiency, whilst also providing tri-axial force information. In addition, the number of inputs and outputs (I/Os) required for the 3-cross bar structure 1612 is less than, for example the 4-cross bar structure 1608, thereby allowing relatively more space for integrated circuits (ICs) and through-silicon vias (TSVs).

FIG. 16 also shows that the embodiment has a functional force range of up to 25 mN. No further deflection 1602 can be seen for forces beyond 25 mN for the 3-cross bar structure 1612 and the 4-cross bar structure 1608. This shows that the force transmission element acts as a mechanical stopper when a force exceeding 25 mN is experienced by the force transmission element, thereby preventing further deformation to protect the electrical and mechanical structures of the tactile sensor.

While various embodiments of the micro-sensory tip have been described in terms of guidewires or catheters, the various embodiments may also be applied for other medical applications involving haptic feedback for prosthetic arms, minimally invasive surgical tools and robotic manipulation of objects, among others.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A micro-sensory tip for use in blood vessels, the micro-sensory tip comprising:
a force transmission element;
at least three force detecting sensors coupled to the force transmission element, each of the at least three force detecting sensors responsive to force applied on the force transmission element, wherein each of the at least three force detecting sensors produces an output representing at least one force component of a three-dimensional Cartesian coordinate system, of the force experienced by the force transmission element, such that the outputs of the at least three force detecting sensors can cover the space of the three-dimensional Cartesian co-ordinate system; and
an active element arrangement coupled to the at least three force detecting sensors, the active element arrangement configured to process the output from the at least three force detecting sensors.

2. The micro-sensory tip of claim 1, wherein the active element arrangement is further configured to produce an output containing vector information of the force experienced by the force transmission element.

3. The micro-sensory tip of claim 2, wherein the active element arrangement is further configured to provide the vector information as a symbol displayable on two-dimensional Cartesian co-ordinate axes in a display unit.

4. The micro-sensory tip of claim 2, wherein the active element arrangement is further configured to provide the vector information as a symbol displayable on three-dimensional Cartesian co-ordinate axes in a display unit.

5. The micro-sensory tip of claim 1, wherein the active element arrangement comprises application specific integrated circuitry.

6. The micro-sensory tip of claim 5, wherein the application specific integrated circuitry comprises a sensor interface and multiplexer coupled to the at least three force detecting sensors, the sensor interface and multiplexer amplifying and multiplexing the output from the at least three force detecting sensors.

7. The micro-sensory tip of claim 6, wherein the application specific integrated circuitry further comprises a data converter coupled to the sensor interface and multiplexer, the data converter converting the output from the at least three force detecting sensors into digital data.

8. The micro-sensory tip of claim 7, wherein the application specific integrated circuitry further comprises an external reader interface coupled to the data converter, the external reader interface for modulating data from the data converter and decoding command signals from an external reader module.

9. The micro-sensory tip of claim 8, wherein the application specific integrated circuitry further comprises a clock unit coupled to synchronise the operation of the sensor interface and multiplexer, the data converter and the external reader interface.

10. (canceled)

11. The micro-sensory tip of claim 1, wherein the at least three force detecting sensors are arranged on one common plane.

12. The micro-sensory tip of claim 11, wherein an angular displacement between adjacent force detecting sensors is such that the at least three force detecting sensors form a Y-shaped arrangement.

13. The micro-sensory tip of claim 1, further comprising a fourth force detecting sensor, wherein the four force detecting sensors are arranged on one common plane.

14. The micro-sensory tip of claim 13, wherein an angular displacement between the force detecting sensors is such that the at least four force detecting sensors form an X-shaped arrangement.

15. The micro-sensory tip of claim 1, further comprising a stylus to couple the force transmission element to the at least three force detecting sensors.

16. The micro-sensory tip of claim 15, further comprising a spacer layer between the force transmission element and the at least three force detecting sensors, the spacer layer having a cavity wherein the stylus is located, the perimeter of the cavity being in proximity to the force transmission element so that the force transmission element comes into contact with the cavity perimeter when the force experienced by the force transmission element exceeds a threshold.

17. (canceled)

18. The micro-sensory tip of claim 1, wherein each of the at least three force detecting sensors is formed from any one or more of the following deformable structures: a nanowire and a nanotube.

19. The micro-sensory tip of claim 1, wherein each of the at least three force detecting sensors comprises a MOSFET formed therein.

20. (canceled)

21. The micro-sensory tip of claim 1, wherein the at least three force detecting sensors are formed of a material selected from the group consisting of polycrystalline silicon, single crystal silicon, silicon-germanium, germanium, gallium arsenide, silicon carbide, carbon, diamond, metal and any combination thereof.

22. (canceled)

23. A guidewire comprising a wire with the micro-sensory tip of claim 1 located at an end of the wire that is introduced inside a blood vessel.

24. A method of forming a micro-sensory tip for use in blood vessels, the method comprising:
providing a force transmission element;
coupling at least three force detecting sensors to the force transmission element, each of the at least three force detecting sensors responsive to force applied on the force transmission element, wherein each of the at least three force detecting sensors produces an output representing at least one force component of a three-dimensional Cartesian coordinate system, of the force experienced by the force transmission element, such that the outputs of the at least three force detecting sensors can cover the space of the three-dimensional Cartesian co-ordinate system; and
coupling an active element arrangement to the at least three force detecting sensors, the active element arrangement configured to process the output from the at least three force detecting sensors.

25. (canceled)
